(19) United States
(12) Patent Application Publication
(10) Pub. No.: US 2024/0425752 A1
(43) Pub. Date: Dec. 26, 2024

(54) BILIRUBIN-COATED RADIO-LUMINESCENT PARTICLES

(71) Applicant: Purdue Research Foundation, West Lafayette, IN (US)

(72) Inventors: You-Yeon Won, West Lafayette, IN (US); Vincenzo John Pizzuti, Follansbee, WV (US); Dhushyanth Viswanath, West Lafayette, IN (US)

(21) Appl. No.: 18/824,290

(22) Filed: Sep. 4, 2024

Related U.S. Application Data
(62) Division of application No. 17/422,177, filed on Jul. 10, 2021.
(60) Provisional application No. 62/795,141, filed on Jan. 22, 2019.

(51) Int. Cl. C09K 11/68; A61K 41/00; B82Y 5/00; B82Y 20/00; B82Y 30/00; B82Y 40/00; C09K 11/02; C09K 11/56

(52) U.S. Cl. CPC C09K 11/684; A61K 41/0057; C09K 11/025; C09K 11/565; B82Y 5/00; B82Y 20/00; B82Y 30/00; B82Y 40/00

(57) ABSTRACT

The present disclosure relates to novel compositions comprising hydrophilic polymer-conjugated bilirubin-coated radio-luminescent particle or particle aggregates, and methods to make and use the novel compositions. A specific novel PEG-BR/CWO NP system provided in this disclosure comprises a CaWO₄ nanoparticle (CWO NP) core encapsulated by a poly (ethylene glycol)-bilirubin conjugate micelle (PEG-BR micelle).

### CROSS-REFERENCE TO RELATED APPLICATIONS

This application is a divisional application of U.S. patent application Ser. No. 17/422,177, filed Jul. 10, 2021, which is a 371 U.S. national phase of PCT international application no. PCT/US20/13054, filed Jan. 10, 2020, which relates to and claims the benefit of priority to U.S. provisional patent application No. 62/795,141, filed Jan. 22, 2019, the contents each of which are incorporated herein by reference in their entireties.

### TECHNICAL FIELD

The present disclosure relates to novel compositions comprising hydrophilic polymer-conjugated bilirubin-coated radio-luminescent particles or particle aggregates, and methods to make and use the novel compositions.

### BACKGROUND

This section introduces aspects that may help facilitate a better understanding of the disclosure. Accordingly, these statements are to be read in this light and are not to be understood as admissions about what is or is not prior art.

Photodynamic therapy (PDT) is a relatively new modality for cancer treatment that has clinical potential. PDT relies on oxygen, light, and a photosensitizer to function. Photosensitizers are compounds that produce cytotoxic reactive oxygen species (ROS) when exposed to specific wavelengths of light, but are otherwise pharmacologically inactive. Because of this activation pathway, PDT typically displays low systemic toxicity and minimal acquired resistance. One major limitation of PDT is that it cannot treat tumors deeper than the surface level because of the short penetration depths of light in tissue. Thus, only tumors of the skin or surface linings of the esophagus, lung, or bladder can be treated.

Therefore, there is need for new compositions and methods in photodynamic therapy (PDT).

### SUMMARY

The present invention provides novel compositions comprising hydrophilic polymer-conjugated bilirubin-coated radio-luminescent particles or particle aggregates, and methods to make and use the novel compositions.

In one embodiment, the present disclosure provides a composition (“PEG-BR/CWO NPs”) comprising:
a radio-luminescent particle or particle aggregate (such as a CaWO₄ nanoparticle (“CWO NP”)); and
hydrophilic polymer-conjugated bilirubin (such as PEGylated bilirubin (“PEG-BR”));
wherein the radio-luminescent particle or particle aggregate is coated with the hydrophilic polymer-conjugated bilirubin.

In another embodiment, the present disclosure provides a method of treating patients with locally advanced primary or metastatic tumors, wherein the method comprises administering a therapeutically effective amount of a composition to the tumor and exposing the tumor to ionizing radiation, wherein the composition comprises:
a radio-luminescent particle or particle aggregate; and
hydrophilic polymer-conjugated bilirubin;
wherein the radio-luminescent particle or particle aggregate is coated with the hydrophilic polymer-conjugated bilirubin.

### BRIEF DESCRIPTION OF THE DRAWINGS

FIG. 1: Schematic Overview of PEG-BR/CWO NP Mechanism of Action. The top of the figure is a schematic diagram for the mechanism of PEG-BR/CWO NPs. The structure of the PEG-BR conjugate is displayed on the lower portion of the figure.

FIG. 2: TEM Micrograph of PEG-BR/CWO NPs. Filtered PEG-BR/CWO NPs in PBS suspension were air-dried onto a TEM grid and negatively stained with 2% uranyl formate. Several images of the particles were taken, and a representative image is displayed in the figure. As is visible in the representative micrograph, filtered PEG-BR/CWO NPs are predominantly comprised of small clusters of CWO NPs (dark particles) encapsulated by PEG-BR (lighter gray region surrounding particle cluster). Scale bar=50 nm. Primary CWO NPs used in this experiment were approximately 40-50 nm in diameter.

FIG. 3. DLS Size Data for PEG-BR NPs (Micelles) and PEG-BR/CWO NPs. PEG-BR micelles and PEG-BR/CWO NPs were suspended at 0.2 mg/mL concentration in PBS (mass of polymer and CWO for micelles and PEG-BR/CWO NPs, respectively) and analyzed using DLS at room temperature. Effective diameters represent average values and error bars represent standard deviation (N=3). Filtered samples were passed through a 450 nm PTFE syringe filter before analysis by DLS. Unfiltered particles were diluted ten-fold in PBS, and filtered particles were analyzed immediately after filtration without dilution. Each histogram plot is a representative example taken from one of the N=3 samples.

FIG. 4. Absorbances and Fluorescences of Unirradiated NPs. CWO NPs, PEG-BR NPs (micelles), and PEG-BR/CWO NPs (filtered as described in the Materials & Methods section) were suspended at 0.1 mg/mL in PBS (based on mass of polymer for PEG-BR and CWO for CWO NPs and PEG-BR/CWO NPs, respectively). Absorbance (left, 4A) and fluorescence (right, 4B, excitation wavelength 200 nm) measurements were performed using a quartz cuvette with 1 cm path length at 1 nm wavelength intervals. PBS was used as the blank reference for absorbance measurements.

FIG. 5. DLS Size Data for Irradiated PEG-BR/CWO NPs. PEG-BR/CWO NPs at a concentration of 0.1 mg/mL (based on CWO) in PBS were irradiated with UV-A light (peak emission at 365 nm) at a fluence of 0.56 J/cm² (one dose) or 1.12 J/cm² (two doses of 0.56 J/cm²) or 8 Gy of 320 kV X-rays (at a dose rate of 2 Gy/min). DLS size measurements were conducted immediately after formulation, and after UV-A/X-ray doses. The increase in effective diameter is indicative of PEG-BR degradation and release of PEG chains, which then causes agglomeration of non-PEGylated BR/CWO NPs in suspension to increase the number of large particles and thus increase the effective diameter of all particles in the sample. Error bars represent standard deviation (N=3). This trend can be seen in the representative histograms presented. As UV exposure dose increases, an increasing number of larger agglomerates are observed via DLS intensity-weighted size histogram output. X-rays cause greater degrees of PEG-BR degradation and subsequent non-PEGylated BR/CWO NP aggregation. Note: Experiment conducted using filtered particles.

FIG. 6. GPC Traces of PEG-BR before and after Irradiation. PEG-BR NPs (micelles) and PEG-BR/CWO NPs in PBS were irradiated with UV-A (0.56 J/cm²) or X-rays (8 Gy). PEG-BR was extracted from these solutions with DCM (CWO NPs removed by centrifugation), dried, and re-dissolved in HPLC-grade THF (1 mg/mL) for GPC analysis (filtered with a 450 nm PTFE filter prior to GPC). Note: Experiment conducted using filtered particles.

FIG. 7. Absorbances and Fluorescences of Irradiated NPs. PEG-BR/CWO NPs (filtered as described in the Materials & Methods section) were suspended at 0.1 mg/mL in PBS (based on mass of CWO). Absorbance (left, A) and fluorescence (right, B, excitation wavelength 200 nm) measurements were performed using a quartz cuvette with 1 cm path length at 1 nm wavelength intervals. PBS was used as the blank reference for absorbance measurements.

FIG. 8. Singlet Oxygen Production Quantification. Singlet Oxygen Sensor Green (SOSG) was diluted in MilliQ water to a concentration of 10 μM in the wells of a 96-well plate containing suspensions of PBS, CWO NPs, and PEG-BR/CWO NPs at a concentration of 0.2 mg/mL (based on CWO NP concentration or equivalent volume of PBS). Two separate sets of samples were prepared for irradiated groups (to measure singlet oxygen production under X-ray) and unirradiated groups (to measure background fluorescence signals as negative controls). Irradiated samples were dosed with 0, 3, or 6 Gy of X-ray at a dose rate of 2 Gy/min. Both sets of samples were kept protected from all other illumination sources until time of fluorescence measurement. Sample wells in irradiated and unirradiated plates were read using 500 nm excitation and 525 nm emission endpoints. N=4 per group for irradiated samples and N=3 per group for unirradiated samples. Single asterisks represent p<0.05 and double asterisks represent p<0.01,calculated using two-tailed student's t-test. PEG-BR/CWO NP groups at 3 Gy and 6 Gy were significantly different (p<0.05) than CWO NP and PBS groups at each dose. Note: Experiment conducted with unfiltered nanoparticles.

FIG. 9. Cell Viability with Exposure to PEG-BR/CWO NPs. Cell viability measured by MTT assay with exposure to PEG-BR/CWO NPs at displayed concentrations (based on CWO NP). HN31 cells were seeded in 96-well tissue culture plates at a density of 1.0×10⁴ cells per well and incubated for 24 hours. MTT cell viability assay was performed at 24 h post treatment. 0 mg/mL represents the negative control for these experiments. All error bars represent standard deviation (N=4). Note: Experiment conducted using unfiltered particles.

FIG. 10. PEG-BR/CWO NP Initial Clonogenic Cell Survival Assay. HN31 cells were seeded in 6 well plates at 0.2×10³ (0 Gy), 0.8×10³ (3 Gy), 1.6×10³ (6 Gy), and 5.0×10³ (9 Gy) in triplicate for each treatment group. Cells were incubated with PBS, PEG-BR micelles (0.2 mg/mL PEG-BR), and PEG-BR/CWO NPs (0.2 mg/mL CWO nanoparticle) for 4 hours prior to X-ray irradiation. Irradiations were performed at 2 Gy/min using a 320 kV X-ray irradiator. Colonies of greater than 50 cells were counted to calculate survival fraction (N=3). Error bars represent standard deviation.

FIG. 11. CWO NP Comparison Clonogenic Cell Survival Assay. HN31 cells were seeded in 6 well plates at 0.2×10³ (0 Gy), 0.8×10³ (3 Gy), 1.6×10³ (6 Gy), and 5.0×10³ (9 Gy) in triplicate for each treatment group. Cells were incubated with PBS, CWO NPs (0.2 mg/mL CWO nanoparticle), and PEG-BR/CWO NPs (0.2 mg/mL CWO nanoparticle) for 4 hours prior to X-ray irradiation. Irradiations were performed at 2 Gy/min using a 320 kV X-ray irradiator. Colonies of greater than 50 cells were counted to calculate survival fraction (N=3). Error bars represent standard deviation.

FIG. 12. Murine HNSCC Xenograft with NP Treatment. Subcutaneous xenografts were produced by inoculation of 1.5×10⁶ HN31 cells in 0.1 mL total volume in Nod rag gamma (NRG) mice (day 0). Intratumoral injection of 100 μL of 10 mg/mL CWO NPs in sterile PBS was conducted in two portions over two days (days 6 and 7, see arrow on graph for first injection) once tumors reached ˜100 mm³; blank PBS was injected in the control (PBS±X-ray only) group. “Sub-therapeutic” (i.e., low-dose) radiation treatments with 320 keV X-rays were conducted on the second day of injection (day 7) and the subsequent day (day 8, 2 Gy each) for a total dose of 4 Gy. Tumors were measured with digital calipers. Tumor volumes for each group are displayed up to the first euthanasia event that occurred in each group. Error bars represent standard error. Asterisk denotes p<0.1 using two-tailed student's t-test between PBS X-ray and PEG-BR/CWO NP+X-ray groups on day 21 (the only point of significant difference, brackets highlight curves being compared). Euthanasia criteria were >20% body weight loss or tumor volume >2,000 mm³. N=8 per treatment group. Note: Unfiltered particles were used for this experiment.

FIG. 13. Murine HNSCC Xenograft with NP Treatment. Kaplan-Meier survival curves were generated for the mice from the study detailed in FIG. 12. Euthanasia criteria were >20% body weight loss or tumor volume >2,000 mm³. N=8 per treatment group. Open circles indicate euthanasia based on criteria other than tumor volume, such as body weight loss, tumor ulceration, and tumor fluid leakage.

FIG. 14. Murine HNSCC Xenograft with NP Treatment. Subcutaneous xenografts were produced by inoculation of 1.5×10⁶ HN31 cells in 0.1 mL total volume in Nod rag gamma (NRG) mice (day 0). Intratumoral nanoparticle treatment injection of 100 μL at 10 mg/mL (based on CWO mass) in sterile PBS was conducted in two portions over two days (days 4 and 5, see arrow on graph for first injection) once tumors reached ˜100 mm³; blank PBS was injected in the control (PBS±X-ray only) group. “Sub-therapeutic” (i.e., low-dose) radiation treatments with 320 keV X-rays were conducted on the second day of injection (day 5) and the subsequent three days (days 6, 7 and 8) for a total dose of 8 Gy (2 Gy each day). Tumors were measured with digital calipers. Tumor volumes for each group are displayed up to the first euthanasia event that occurred in each group. Error bars represent standard error. Euthanasia criteria were >20% body weight loss or tumor volume >2,000 mm³. N=8 for PBS and CWO NP, and N=9 for the other groups. Note: Unfiltered particles were used for this experiment.

FIG. 15. Murine HNSCC Xenograft with NP Treatment. Kaplan-Meier survival curves were generated for the mice from the study detailed in FIG. 14. Euthanasia criteria were >20% body weight loss or tumor volume >2,000 mm³. N=8 for PBS and CWO NP, and N=9 for the other groups. Open circles indicate euthanasia based on criteria other than tumor volume, such as body weight loss, tumor ulceration, and tumor fluid leakage.

FIG. 16. H&E Stained Histology Sections of Major Organ and Tumor Tissues. From the mouse efficacy study discussed in FIG. 9 and FIG. 10, major organ and tumor tissues were excised and fixed in 10% neutral buffered formalin. Then, fixed tissues were embedded in paraffin blocks, sectioned, stained using hematoxylin and eosin (H&E), and mounted onto microscope slides for imaging. Digital scans of the slides were performed, and representative images of tissue sections are displayed above. (A) HN31 xenograft tumor sections from PBS, CWO NP, and PEG-BR/CWO NP+X-ray-treated groups. Top images are from regions of higher cell viability and bottom images display areas of higher damage. (B) Major tissue sections from same animals displayed in (A). Liver, lung, heart, spleen, brain, and kidney sections are displayed for each. In the lungs of each animal, dense or nodules are observed in the bottom right corner, indicating lung metastasis has occurred in each animal. In addition, the spleen from each animal was markedly enlarged upon excision. Scale bars=200 μm.

FIG. 17. TEM Micrograph of PEG-BR/CWO/PTX NPs. Filtered PEG-BR/CWO NPs co-loaded with a chemo drug paclitaxel (PTX) (“PEG-BR/CWO/PTX NPs”) in PBS suspension were air-dried onto a TEM grid and negatively stained with 2% uranyl acetate. Several images of the particles were taken, and a representative image is displayed above. As is visible in the representative micrograph, filtered PEG-BR/CWO/PTX NPs are predominantly comprised of small clusters of CWO NPs (dark particles) and PTX (dark ring around each particle) encapsulated by PEG-BR (lighter gray region surrounding particle cluster). Scale bar=50 nm. Primary CWO NPs used in this experiment were approximately 40-50 nm in diameter.

FIG. 18. DLS Size Data of PEG-BR/CWO/PTX NPs. PEG-BR/CWO/PTX NPs were suspended in PBS at 0.2 mg/mL (based on mass of CWO). For DLS analysis, unfiltered particles were diluted ten-fold in PBS (Left, A), and filtered particles were analyzed immediately after filtration (with a 450 nm PTFE syringe filter) without dilution (Right, B). The mean hydrodynamic diameters of the unfiltered and filtered particles were determined to be 487 and 158 nm, respectively.

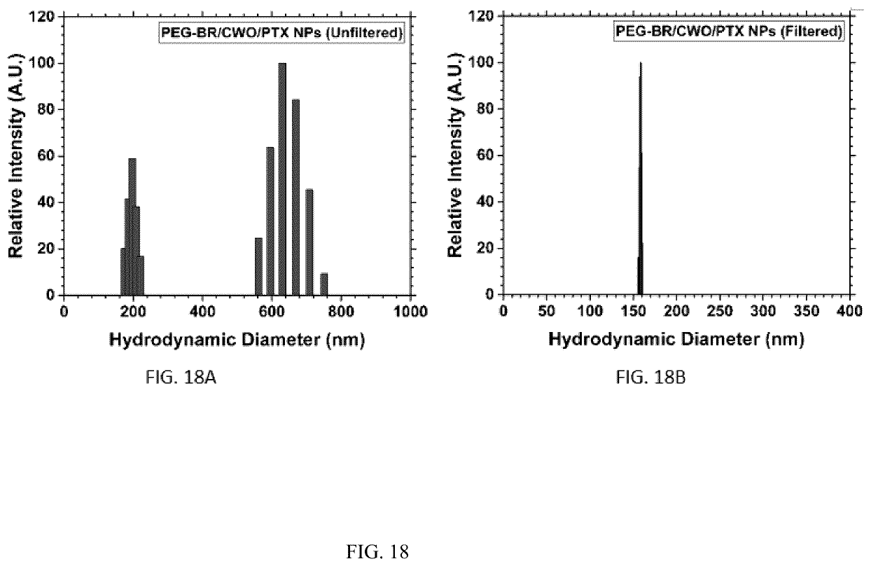

### DETAILED DESCRIPTION

For the purposes of promoting an understanding of the principles of the present disclosure, reference will now be made to embodiments illustrated in drawings, and specific language will be used to describe the same. It will nevertheless be understood that no limitation of the scope of this disclosure is thereby intended.

In the present disclosure the term “about” can allow for a degree of variability in a value or range, for example, within 10%, within 5%, or within 1% of a stated value or of a stated limit of a range.

In the present disclosure the term “PEG-BR/CWO NPs” means poly (ethylene glycol)-conjugated bilirubin (PEG-BR)-encapsulated CaWO₄ nanoparticles (CWO NPs).

In the present disclosure the interchangeable term “coated” and “encapsulated” refer to using a micelle formed by PEG-BR to encapsulate a nanoparticle such as a CWO nanoparticle within the micelle. Therefore, there may or may not have some space/distance between the surface of the nanoparticle and the PEG-BR polymer conjugate.

In the present disclosure the term “radiation” refers to ionizing-radiation or non-ionizing radiation. Ionizing radiation is radiation that carries enough energy to liberate electrons from atoms or molecules, thereby ionizing them. Ionizing radiation may include but is not limited to short-wavelength ultraviolet (UV) light, X-rays, γ rays, electrons, protons, neutrons, ions, or any combination thereof. Non-ionizing radiation refers to any type of electromagnetic radiation that does not carry enough energy per quantum (photon energy) to ionize atoms or molecules—that is, to completely remove an electron from an atom or molecule. Non-ionizing radiation may include but is not limited to long-wavelength UV, visible, or infrared (IR) light, or any combination thereof. Non-ionizing radiation may be generated by a laser or lamp-type source, and may be delivered directly or by using a fiber optic to the intended delivery site.

Photodynamic therapy (PDT) has shown potential as a cancer treatment modality, but its clinical application is limited due to its visible light activation since light cannot penetrate tissues well. Additionally, combination therapies utilizing PDT and radiotherapy have shown clinical promise in several cancers but are limited again by light penetration and the need for selective photosensitization of the treatment area.

To address this issue, this disclosure provides a novel PEG-conjugated bilirubin-encapsulated CaWO₄ nanoparticle (PEG-BR/CWO NP) system that acts as an X-ray inducible PDT platform. As previously reported, BR is capable of photosensitizing cells to light, making them more susceptible to damage and death from light exposure. This photodynamic activity is due to the production of reactive oxygen species (ROS), when BR is exposed to UV-A and visible-spectrum wavelengths of light, predominantly singlet oxygen (¹O₂). This singlet oxygen exerts the majority of the therapeutic effects in photodynamic therapy. Further, because X-ray photons have much better penetration depths into tissue, they can overcome the limitations of visible light. Thus, this system may be used to treat locally advanced primary or recurrent lesions anywhere within the body. Additionally, because the platform is X-ray activated, the system acts as a potentiator for combined radio and photodynamic therapy, a combination that has shown promising results.

One specific example of cancer type that can benefit from radiotherapy and PDT combination is head and neck squamous cell carcinoma (HNSCC). HNSCC is the 6^{th} most common cancer worldwide, and the overall 5-year survival rate is around 50% for all HNSCC patients. It has relatively high incidence of recurrence post-radiotherapy, with the rate of recurrence up to 60% for local failure and 30% for distant failure. This is an issue considering that radiation therapy is a primary treatment modality for most cases of HNSCC. The combination of PDT and radiation therapy is expected to show improved clinical responses in patients with HNSCC since the two therapies operate through separate ROS generation mechanisms, but this strategy is still limited in that PDT is only an option for tumors on surfaces of the nose, mouth, and throat. The PEG-BR/CWO NPs overcome this because their X-ray activation allows the system to be actuated even below the surfaces of tissues, allowing for radiation therapy and PDT combinations in large or deep-seated tumors.

Typically, therapeutics used to treat cancers are systemically administered, causing off-target toxicities. Intratumoral injection is a clinically viable delivery method for cancer therapies that helps to overcome this limitation because the therapeutic regimen is only applied to the diseased tissue. In the case of PEG-BR/CWO NPs, intratumoral injection ensures good localization of treatment since the therapeutic effects are only activated by the external X-ray source, which is focused on the tumor itself. In this way, this system is specific for diseased tissues and minimizes off-target toxicity.

A specific novel PEG-BR/CWO NP system provided in this disclosure comprises a CaWO₄ nanoparticle (CWO NP) core encapsulated by a poly (ethylene glycol)-bilirubin conjugate micelle (PEG-BR micelle). When conjugated to PEG, bilirubin can intramolecularly hydrogen bond, creating a hydrophobic domain that drives the assembly of micelles in aqueous medium. It has been reported that when exposed to UV-A/blue wavelengths of light, bilirubin undergoes rearrangement that disrupts the extensive intramolecular hydrogen bonding network, thus eliminating its hydrophobicity; this loss of hydrophobic character ultimately causes the PEG-BR micelles to dissociate. This disclosure provides new compositions comprising hydrophilic polymer-conjugated bilirubin-coated radio-luminescent particles or particle aggregates by using PEG-BR micelles, taking advantage of unexpected photo-activatable properties different from those described above, with a focus on their use as dual photo-and radio-sensitizing agents. With PEG-BR/CWO NPs, this disclosure provides a new application for PEG-BR when combined with the radio-luminescent properties of CWO NPs. Under X-ray/UV-A exposure, PEG-BR/CWO NPs employ the cleavage of the PEG-BR molecules into the PEG and BR precursors (instead of the previously described PEG-BR micelle dissociation), in addition to bilirubin's innate photo-sensitizing capabilities, to facilitate the activation of combined PDT and radiation therapy.

The present disclosure relates to novel compositions comprising hydrophilic polymer-conjugated bilirubin-coated radio-luminescent particles or particle aggregates, and methods to make and use the novel compositions.

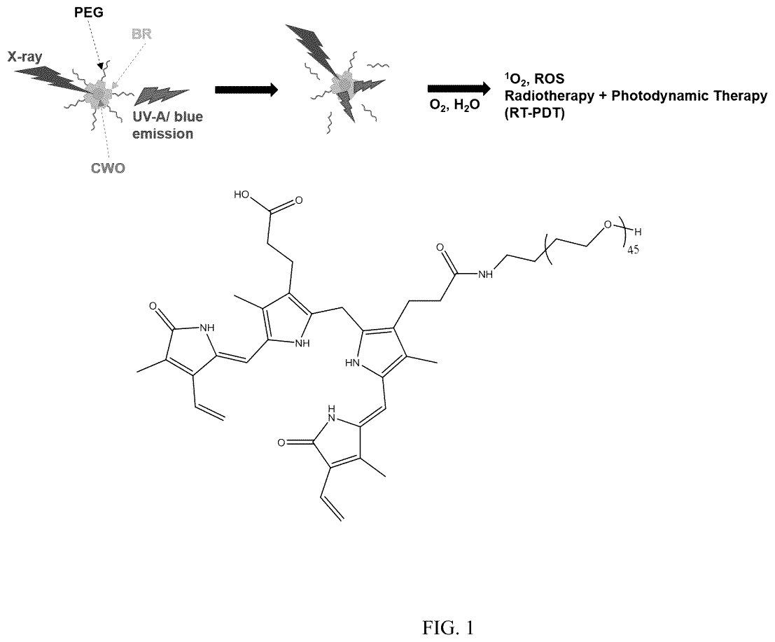

FIG. 1 explains the concept of the novel hydrophilic polymer-conjugated bilirubin-coated radio-luminescent particle or particle aggregate. The top of the figure is a schematic diagram for the mechanism by which the PEG-BR/CWO NP works. The structure of the PEG-BR conjugate is also displayed in the lower figure. Specifically, bilirubin photodynamic nanoparticles (“PEG-BR/CWO NPs”) are thought to potentiate photodynamic therapy under X-ray irradiation through distinct steps. X-ray exposure causes CaWO₄ (CWO) nanoparticles at the core of the PEG-BR/CWO NPs to emit UV-A and blue light. The X-ray and UV-A/blue light combination causes the degradation of PEG-BR into PEG and BR, leading to detachment of PEG chains from PEG-BR/CWO NPs and leaving only a monolayer of BR on the CWO NP surface. After this dissociation of the steric PEG layer, CWO will continue emitting UV-A/blue light, which will interact with the surface-exposed BR in the BR/CWO NPs. This excited BR can interact with intra- and extracellular molecular oxygens, and reactive oxygen species (ROS) are produced, predominantly singlet oxygen (¹O₂). Singlet oxygen effects combined with X-ray cellular damage improve the efficacy of X-ray treatments for cancers.

In one embodiment, the present disclosure provides a composition comprising:
a radio-luminescent particle or particle aggregate; and
hydrophilic polymer-conjugated bilirubin;
wherein the radio-luminescent particle or particle aggregate is coated with the hydrophilic polymer-conjugated bilirubin.

In one embodiment, the radio-luminescent particle or particle aggregate emits light in the wavelength range of 350-700 nm, 350-600 nm, 350-550 nm, 400-700 nm, 400-600 nm, or 400-550 nm under ionizing radiation that causes bilirubin to produce reactive oxygen species. In one aspect, the wavelength range is 400-550 nm.

In one embodiment, the radio-luminescent particle or particle aggregate comprises a radio-luminescent nanoparticle or nanoparticle aggregate, wherein the mean diameter of said radio-luminescent nanoparticle is in the range between about 1 nm and about 50,000 nm in its unaggregated state.

In one embodiment, the radio-luminescent particle or particle aggregate comprises a metal tungstate material (Mₓ(WO₄)_{y}) which comprises a metal compound (M) selected from the “Alkaline Earth Metal”, “Transition Metal” or any combination thereof.

In one embodiment, the radio-luminescent particle or particle aggregate comprises calcium tungstate (CaWO₄), iron tungstate (FeWO₄), manganese tungstate (MnWO₄), or a combination thereof.

In one embodiment, the radio-luminescent particle or particle aggregate comprises a metal molybdate material (Mₓ(MoO₄)_{y}) which comprises a metal compound (M) selected from the “Alkaline Earth Metal”, “Transition Metal” or any combination thereof.

In one embodiment, the radio-luminescent particle or particle aggregate comprises calcium molybdate (CaMoO₄), iron molybdate (FeMoO₄), manganese molybdate (MnMoO₄), or a combination thereof.

In one embodiment, the radio-luminescent particle or particle aggregate comprises zinc oxide (ZnO), zinc sulfide (ZnS), or a combination thereof.

In one embodiment regarding the radio-luminescent particle or particle aggregate-containing composition, wherein the composition further comprises one or more hydrophobic chemotherapeutic drugs, wherein the radio-luminescent particle or particle aggregate and the hydrophobic chemotherapeutic drug are co-encapsulated within a capsule formed by the hydrophilic polymer-conjugated bilirubin. Said hydrophobic chemotherapeutic drug can be, but not limited to, paclitaxel, docetaxel, cabazitaxel, cisplatin, carboplatin, oxaliplatin, nedaplatin, doxorubicin, daunorubicin, epirubicin, idarubicin, gemcitabine, etanidazole, 5-fluorouracil, methotrexate, any salt or derivative thereof, or any combination thereof. In one aspect, said hydrophobic chemotherapeutic drug is paclitaxel, docetaxel, cabazitaxel, any salt or derivative thereof, or any combination thereof. In one aspect, said hydrophobic chemotherapeutic drug is paclitaxel. In one aspect, the hydrophobic chemotherapeutic drug has a water solubility less than 200 mg/mL, less than 100 mg/mL, 50 mg/mL, or 25 mg/mL at room temperature. In one aspect, the hydrophobic chemotherapeutic drug has a water solubility in the range of about 0.00001-200 mg/mL, 0.00001-100 mg/mL, 0.00001-50 mg/mL, 0.00001-25 mg/mL, 0.00005-200 mg/mL, 0.00005-100 mg/mL, 0.00005-50 mg/mL, 0.00005-25 mg/mL, 0.0001-200 mg/mL, 0.0001-100 mg/mL, 0.0001-50 mg/mL, or 0.0001-25 mg/mL at room temperature.

In one embodiment regarding the radio-luminescent particle or particle aggregate-containing composition, wherein the composition further comprises one or more pharmaceutically acceptable carriers, diluents and/or excipients.

In one embodiment, the hydrophilic polymer-conjugated bilirubin forms a self-assembled structure in water, wherein the radio-luminescent particle or particle aggregate is encapsulated within the hydrophobic subdomain of the self-assembled structure formed by the bilirubin component.

In one embodiment, the hydrophilic (water-soluble) polymer comprises a monomer selected from the group consisting of ethylene glycol, ethylene oxide, vinyl alcohol, oxazoline, acrylic acid, methacrylic acid, acrylamide, styrene sulfonate, saccharide, imine, vinyl pyrrolidone, vinyl pyridine, and lysine.

In one embodiment, the hydrophilic polymer-conjugated bilirubin is poly(ethylene glycol)(PEG)-conjugated bilirubin.

In one embodiment, the composition has a radiation sensitizer enhancement ratio (SER, defined as the ratio of the radiation dose at 10% clonogenic survival in the absence of radio-luminescent particles relative to the radiation dose at 10% survival in the presence of radio-luminescent particles) greater than 1.1 when measured using a radiation with a peak energy in the range between about 0.1 MeV and about 6.0 MeV at a radio-luminescent particle concentration less than or equal to about 0.2 mg/mL in tumor cell cultures.

In another embodiment, the present disclosure provides a method of treating a disease responsive to any composition of this disclosure, wherein the method comprises administering any composition of this disclosure directly into the diseased site, and exposing the diseased site to ionizing radiation, wherein the ionizing radiation comprises UV light, X-rays, γ rays, electrons, protons, neutrons, ions, or any combination thereof. In one aspect, the disease is a cancer. In one aspect, the cancer involves solid tumors. In one aspect, the tumors are related to head and neck, lung, brain, muscle, bone, stomach, liver, pancreatic, renal, colon, rectal, prostate, breast, gynecological, or cervical tissues.

In another embodiment, the present disclosure provides a method of using any composition of this disclosure in treating patients with locally advanced primary or metastatic tumors, wherein the method comprises administering a therapeutically effective amount of composition to the tumor and exposing the tumor to ionizing radiation.

In one embodiment regarding the method of using any composition of this disclosure, the ionizing radiation comprises UV light, X-rays, γ rays, electrons, protons, neutrons, ions, or any combination thereof.

In one embodiment regarding the method of using any composition of this disclosure, said tumors are solid tumors.

In one embodiment regarding the method of using any composition of this disclosure, said tumors are related to head and neck, lung, brain, muscle, bone, stomach, liver, pancreatic, renal, colon, rectal, prostate, breast, gynecological, or cervical tissues.

In one embodiment regarding the method of using any composition of this disclosure, the composition is delivered to the tumor via intratumoral injection in order to limit toxicity in normal tissues.

In one embodiment, the polymer-conjugated bilirubin material disclosed in the present disclosure may be further functionalized with folic acid. In one aspect, the folic acid functionalized polymer-conjugated bilirubin material may enhance the cellular uptake of the composition, or may have the potential to be used for systemic delivery of the composition in cancer treatment.

### Materials and Methods

### Synthesis and Characterization of PEGylated Bilirubin (PEG-BR)

The poly (ethylene glycol)-conjugated bilirubin (PEG-BR) was synthesized as previously described, with some modification. See Lee, Y.; Lee, S.; Lee, D. Y.; Yu, B.; Miao, W.; Jon, S., Multistimuli-Responsive Bilirubin Nanoparticles for Anticancer Therapy. Angewandte Chemie International Edition 2016, 55 (36), 10676-10680. Briefly, 0.5 mmol of bilirubin (BR) and 0.5 mmol of N,N′-dicyclohexylcarbodiimide (DCC) with 0.5 mmol of N-hydroxysuccinimide (NHS) were dissolved in 5 mL of dimethyl sulfoxide (DMSO) and allowed to stir for 10 minutes at room temperature. Then, 0.2 mmol of HO-PEG2000-NH₂ (Laysan Bio) and 150 μL of triethylamine (TEA) was added to the mixture and allowed to stir for 4 hours at room temperature under a nitrogen or argon atmosphere (synthesis vessel covered to protect it from light). Then 45 mL of methanol was added to the reaction vessel to precipitate free bilirubin (unconjugated BR). The mixture was then centrifuged at 5,000 rpm for 10 minutes, and the supernatant was removed for processing while the precipitate was discarded. The supernatant was then syringe filtered using a 450 nm PTFE filter to remove residual free BR and was then placed under vacuum to concentrate the mixture. The mixture was then dialyzed against Milli-Q filtered water for 2 days using a regenerated cellulose membrane with a MWCO of 1 kDa. The resultant suspension was then lyophilized, and the powder analyzed using ¹H-NMR. For NMR characterization, 5 mg of as-synthesized PEG-BR was dissolved in deuterated DMSO (DMSO-d6) and the spectrum acquired on a Bruker DRX-500 machine.

### Formulation of PEG-BR Micelles and PEG-BR Photodynamic Nanoparticles (PEG-BR/CWO NPs)

For PEG-BR micelles, 10 mg of PEG-BR was dissolved in chloroform and subsequently dried under argon or nitrogen gas and then allowed to dry under vacuum for 4 hours. Then, 10 mL of phosphate buffered saline (PBS) was added to the dried PEG-BR and then sonicated for 5 minutes. The resulting suspension was filtered with a 450 nm PTFE syringe filter.

For PEG-BR photodynamic nanoparticles (PEG-BR/CWO NPs), 20 mg of PEG-BR (synthesized as described previously, with some modification. Lee, Y.; Lee, S.; Lee, D. Y.; Yu, B.; Miao, W.; Jon, S., Multistimuli-Responsive Bilirubin Nanoparticles for Anticancer Therapy. Angewandte Chemie International Edition 2016, 55 (36), 10676-10680) was dissolved in 3.9 g of N,N-dimethylformamide (DMF). Then, 50 μL of 10 mg/mL calcium tungstate nanoparticles was added to the solution. The vial was placed in a sonication bath and an overhead disperser was placed into the mixture and set to rotate at 10,000 rpm. After the initiation of stirring, 2.1 mL of PBS was added to the suspension and allowed to mix for 5 minutes. The resultant solution was removed from the setup and centrifuged at 5,000 rpm for 10 minutes. The supernatant was removed, and the pellet resuspended in PBS with an amount corresponding to the desired final concentration. This mixture was then vortexed for 30 seconds to complete the resuspension. These particles were then filtered with a 450 nm PTFE syringe filter.

### NP Size Characterizations by Transmission Electron Microscopy (TEM) and Dynamic Light Scattering (DLS)

TEM was conducted on PEG-BR/CWO NPs to visualize the as-formulated particles. Images were taken using a Tecnai T20 instrument using 2% uranyl formate as a negative staining agent.

The hydrodynamic sizes of NPs were measured by DLS. For DLS preparation, PEG-BR/CWO NPs were diluted to a concentration of 0.25 mg/mL (based on CaWO₄, CWO) and filtered as described above. N=3 separate batches were prepared and measured.

### Absorbance and Fluorescence Characterizations of PEG-BR, PEG-BR/CWO and CWO NPs

Absorbance and fluorescence measurements were conducted using a Cary 100 Bio UV-Vis Spectrophotometer and a Cary Eclipse Fluorescence Spectrophotometer, respectively. Measurements were performed in a quartz cuvette with 1 cm path length. All samples were prepared in PBS at an active ingredient concentration of 0.1 mg/mL (based on mass of CWO for CWO and PEG-BR/CWO NPs, or based on mass of polymer for PEG-BR NPs). PEG-BR and PEG-BR/CWO NP samples were filtered as described above. For absorbance measurements, PBS was used as the blank reference. Samples were vortexed for 10 seconds prior to measurement to ensure homogeneity.

Absorbance and fluorescence measurements were also performed similarly on PEG-BR, PEG-BR/CWO and CWO NPs after irradiation with UV-A light (using a UVP's B-100AP lamp with a peak wavelength of 365 nm at a total UV-A fluence of 0.56 or 61.6 J/cm²) or X-rays (using a X-RAD 320 irradiator with a peak photon energy of 320 kV at a total dose of 8 Gy and a dose rate of 2 Gy/min).

### UV/X-Ray Dissociation Characterization of PEG-BR/CWO NPs by DLS

A UV-A lamp (peak emission at 365 nm) was used to illuminate filtered PEG-BR/CWO NPs formulated as described above at a final concentration of 0.1 mg/mL (based on CWO) for a total UV fluence of 0.56 J/cm² (or 1.12 J/cm² for the sample exposed to two subsequent doses). DLS size measurements were conducted immediately after formulation, after one UV dose, and after two UV doses (N=3 separate experiments). Identical measurements were also performed on PEG-BR/CWO NPs after irradiation with 8 Gy of 320-kV X-rays at a dose rate of 2 Gy/min (N=3).

### Gel Permeation Chromatography (GPC) Characterization of PEG-BR Irradiated ith UV-A Light or X-Rays

PEG-BR NP and PEG-BR/CWO NP suspensions were prepared in PBS at an active ingredient concentration of 0.25 mg/mL (based on mass of CWO for PEG-BR/CWO NPs or based on mass of polymer for PEG-BR NPs). Afterwards, PEG-BR NPs and PEG-BR/CWO NPs were filtered as described above. PEG-BR NPs and PEG-BR/CWO NPs were irradiated with UV-A or X-rays as described above. Subsequently, 5 mL of dichloromethane (DCM) was added to 2 mL of the aqueous PEG-BR NP or PEG-BR/CWO NP suspension, and the mixture was vortexed for 2 minutes. The resulting emulsion was centrifuged at 5,000 rpm for 10 minutes. 4 mL of the DCM phase (bottom layer) was carefully collected and allowed to dry overnight in a vacuum oven. The dried PEG-RB was re-dissolved in 200 μL of HPLC-grade tetrahydrofuran (THF). The solution was vortexed and sonicated to ensure complete dissolution of the polymer. The solution was filtered with a 450 nm PTFE syringe filter.

GPC measurements were performed on a Waters Breeze GPC system equipped with an isocratic HPLC pump, Styragel HR 4 (10⁴ Å pore size) and Ultrastyragel (500 Å pore size) columns (7.8×300 mm per column), and a differential refractometer. THF was used as the mobile phase at 30° C. at a flow rate of 1 mL/min. 20 μL of the PEG-BR solution in THF was injected into the GPC instrument, and in each run, the RI output was recorded for 25 minutes. Unirradiated PEG-NH_{2,} BR, and PEG-BR were also characterized by GPC for comparison.

### Singlet Oxygen Production Quantification

Singlet Oxygen Sensor Green (SOSG, ThermoFisher) was dissolved into a methanol stock solution at a concentration of 5 mM. Then, aqueous dilutions of SOSG to a concentration of 10 μM and CWO NPs or PEG-BR/CWO NPs to a concentration of 0.1 mg/mL (based on CWO NP concentration) were loaded into the wells of a 96-well plate. Two separate sets of samples were prepared for irradiated groups (to measure singlet oxygen production under X-ray) and unirradiated groups (to measure background fluorescence signals as negative controls). Irradiated samples were dosed with 3 or 6 Gy of X-ray at a dose rate of 2 Gy/min (320 kV XRAD-320,Precision X-ray). Both sets of samples were kept protected from all other illumination sources until time of fluorescence measurement. Sample wells in irradiated and unirradiated plates were read using a Bio-RAD Microplate Reader-550 using 500 nm excitation and 525 nm emission endpoints (N=4 per group).

HN31 cells were used as a cellular model for head and neck squamous cell carcinoma (HNSCC). HN31 cells were cultured in Dulbecco's Modified Eagle Medium (DMEM) supplemented with 10% (v/v) fetal bovine serum (FBS) and 0.1% L-glutamine (Gibco Life Technologies) (as recommended by American Type Culture Collection (ATCC)) in a humidified incubator with 5% CO₂ at 37.0° C.

### MTT Cell Viability Assay

HN31 cells were seeded in a 96-well tissue culture plate at a density of 0.5×10⁴ cells per well and incubated for 24 hours at 37.0° C. in a 5% CO₂ incubator prior to exposure to CWO NPs. Cells were then treated with various concentrations of PEG-BR-coated and uncoated CWO NPs (0.1, 0.2, 0.5 and 1.0 mg CWO per mL solution) (N=4). After 24 hours of incubation, 10 μL of the MTT reagent (Sigma) was added to each well and incubated for additional 4 hours. Resultant formazan crystals were dissolved by first removing all liquid in each well and then adding 150 μL of DMSO (Sigma) to each well. The absorbances at 570 nm and 630 nm (for background subtraction) were immediately measured using a microplate reader (BIO-RAD Microplate Reader-550). The wells containing cells (that had not been treated with CWO NPs) in the medium with the MTT reagent were used as controls for 100% viability reference.

### Clonogenic Cell Survival Assays

The clonogenic cell survival assay was conducted as previously described; see Franken, N. A. P.; Rodermond, H. M.; Stap, J.; Haveman, J.; van Bree, C., Clonogenic assay of cells in vitro. Nature Protocols 2006, 1, 2315. Briefly, HN31 cells were grown in a T-25 cell culture flask until they reached ˜80% confluence. After this, the growth medium was removed, and the adherent cells were washed with PBS (Gibco Life Technologies). Cells were then detached from the plates by treatment with TrypLE™ Express (1×) solution for 4-6 minutes at 37.0° C. Detached cells, suspended in growth medium/TrypLE Express mixture, were centrifuged at 300×g for 5 minutes at room temperature. The cell pellet was resuspended in a minimal amount of growth medium (2-3 mL), and the cells were counted using a hemocytometer.

Cells were then seeded into 6-well plates at densities varying with planned radiation dose, as follows: 0.2×10³ cells/well for 0 Gy, 0.8×10³ cells/well for 3 Gy, 1.6×10³ cells/well for 6 Gy, and 5.0×10³ cells/well for 9 Gy. Three experimental groups were tested with N=3 wells/group: PBS-treated+X-ray, PEG-BR NPs (micelles)+X-ray, and PEG-BR/CWO NPs+X-ray. PEG-BR micelles were diluted in growth medium to a concentration of 0.2 mg/mL (based on polymer concentration), PEG-BR/CWO NPs were diluted in growth medium to 0.2 mg/ml (based on CWO concentration), and PBS was added to an equivalent volume fraction as the experimental groups in growth medium. These prepared doses were added to their respective wells and allowed to incubate at 37.0° C. with the cells for 4 hours and the plates were then exposed to the appropriate dose of X-ray radiation at a dose rate of 2 Gy/minute (320 kV XRAD-320,Precision X-ray). Irradiated cells were cultured for 14 days. Colonies resulting from radio-resistant cells were stained with Crystal Violet. Colonies of more than 50 daughter cells in culture were counted (N32 3). Results were compared with unirradiated controls to calculate survival fraction.

### Murine HN31 Xenograft Efficacy Evaluation

Female Nod rag gamma (NRG) mice (8 weeks old) were housed in a pathogen-free environment including standard cages with free access to food and water and an automatic 12 h light/dark cycle. The mice were acclimated to the facility for 1 week prior to beginning experiments, and all animals were cared for according to guidelines established by the American Association for Accreditation of Laboratory Animal Care (AAALAC). Subcutaneous HNSCC xenografts were produced by inoculation of 1.5×10⁶ HN31 cells in 0.1 mL total volume of a serum free medium containing 50% Matrigel (BD Bioscience). Intratumoral nanoparticle injection at 10 mg/cc tumor of CWO NP in sterile PBS was conducted once tumors reached ˜100 mm³, approximately 6 days after inoculation, and split into two equal injections on consecutive days. For this study, the following treatment groups were used: PEG-BR/CWO NP+X-Ray, CWO NP+X-Ray, and PBS+X-Ray; PEG-BR/CWO NP, CWO NP, and PBS. Radiation treatments were conducted the second day of injection and the subsequent days (2 Gy each day) for a total of 4 or 8 Gy at a dose rate of 2 Gy/min using a 320 kVp laboratory X-ray irradiator (X-RAD 320, Precision X-ray, North Branford, CT). Tumors were measured with digital calipers in three dimensions: length (L), width (W), and height (H). Tumor volumes were calculated using V=(L×W×H)×π/6. N=8 per group. Euthanasia criteria were >20% body weight loss or tumor volume >2,000 mm³. Mice were euthanized via spinal dislocation under anesthesia. Tumors were excised and weighed post euthanasia. All major organs (brain, heart, lungs, kidneys, spleen, liver) and tumors were excised and placed in 10% neutral-buffered formalin phosphate. Representative animal organs from each treated group were then embedded in paraffin, sectioned, stained with standard H&E staining, and digitized with a brightfield digital microscope camera at a zoom of 20×.

### Formulation of PEG-BR/CWO/PTX NPs

For PEG-BR/CWO/PTX NPs, 20 mg of PEG-BR and a designated amount of PTX were co-dissolved in 3.9 g of N,N-dimethylformamide (DMF). Then, 50 μL of 10 mg/mL calcium tungstate nanoparticles (synthesized as described in Lee, J.; Rancilio, N.; Poulson, J.; Won, Y., Block Copolymer-Encapsulated CaWO₄ Nanoparticles: Synthesis, Formulation, and Characterization. ACS Applied Materials & Interfaces 2016, 8 (13), 8608-8619) was added to the solution. The vial was placed in a sonication bath and an overhead disperser was placed into the mixture and set to rotate at 10,000 rpm. After the initiation of stirring, 2.1 mL of PBS was added to the suspension and allowed to mix for 5 minutes. The resultant solution was removed from the setup and centrifuged for 10 minutes at 5,000 rpm. The supernatant was removed, and the pellet resuspended in PBS with an amount corresponding to the desired final concentration. This mixture was then vortexed for 30 seconds to complete the resuspension. These particles were then filtered with a 450 nm PTFE syringe filter as required.

### NP Size Characterizations by Transmission Electron Microscopy (TEM) and Dynamic Light Scattering (DLS)

TEM was conducted on PEG-BR/CWO/PTX NPs to visualize the as-formulated particles. Images were taken using a Tecnai T20 instrument using 2% uranyl acetate as a negative staining agent.

The hydrodynamic sizes of PEG-BR/CWO/PTX NPs were measured by DLS. For DLS preparation, PEG-BR/CWO/PTX NPs were diluted to a concentration of 0.25 mg/mL (based on CWO) and filtered as described above.

### Determination of PTX Loading Efficiency

The PEG-BR/CWO/PTX NP suspension (prepared as described above) was centrifuged at 5,000 rpm for 10 minutes, and the supernatant was discarded. Subsequently, the NPs were re-suspended in 2 mL of PBS to a final concentration of 0.25 mg/mL (based on mass of CWO). 2 mL of dichloromethane (DCM) was added to the aqueous NP suspension, and the resulting emulsion was vortexed to extract PTX with DCM. The emulsion was then centrifuged at 5,000 rpm for 10 minutes, and the DCM phase (bottom layer) was collected and dried overnight in a vacuum oven. The dried residue was dissolved in 200 μL of HPLC-grade acetonitrile (ACN) for HPLC analysis.

Reversed phase HPLC was carried out using an Agilent HPLC/UV system equipped with a Zorbax C-18 5-μm column. A water/ACN (45:55 by volume) mixture (containing 0.1 vol. % formic acid) was used as the mobile phase at an isocratic flowrate of 1 mL/min. 10 μL of the PTX solution in ACN was injected into the HPLC system. Calibration standards were prepared in the range of PTX concentration from 0.00625 to 1.0 mg/mL. Each sample was run for 7 minutes with PTX eluting at ˜5 minutes. The concentration of PTX was estimated from the area of the peak in comparison with a predetermined calibration curve of peak area vs. concentration.

### Results and Discussion

Bilirubin photodynamic nanoparticles (“PEG-BR/CWO NPs”) are thought to potentiate photodynamic therapy under X-ray irradiation through distinct steps. X-ray exposure causes CaWO₄ (CWO) nanoparticles at the core of the PEG-BR/CWO NPs to emit UV-A and blue light. The X-ray and UV-A/blue light combination causes the degradation of PEG-BR into PEG and BR, leading to detachment of PEG chains from PEG-BR/CWO NPs and leaving only a monolayer of BR on the CWO NP surface. After this dissociation of the steric PEG layer, CWO will continue emitting UV-A/blue light, which will interact with the surface-exposed BR in the BR/CWO NPs. This excited BR can interact with intra-and extracellular molecular oxygens, and reactive oxygen species (ROS) are produced, predominantly singlet oxygen (¹O₂). Singlet oxygen effects combined with X-ray cellular damage can potentially improve the efficacy of X-ray treatments for cancers. This mechanism is outlined below in FIG. 1.

### Synthesis and Characterization of PEG-BR/CWO NPs

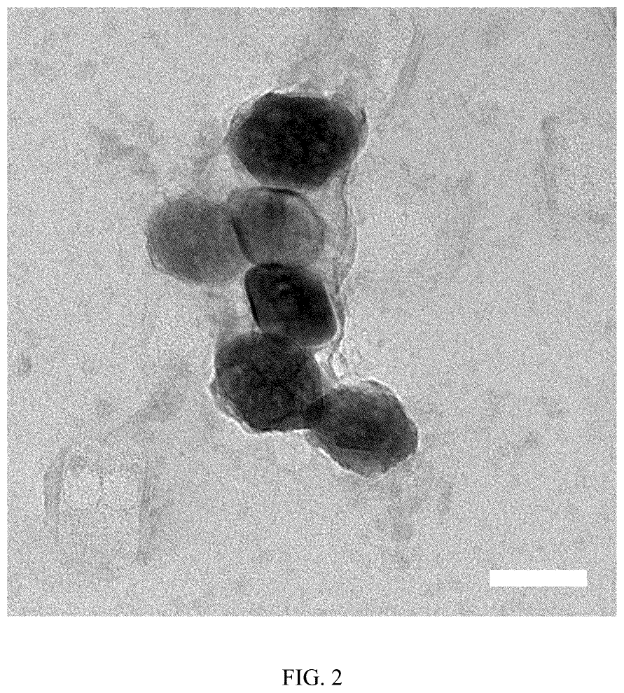

PEG-BR was synthesized from an amine-PEG precursor. The product was then purified, and the resultant compound was characterized via ¹H-NMR to confirm the structure of the product. PEG-BR was then used to encapsulate CaWO₄ nanoparticles (CWO NPs) as described in the Materials and Methods. PEG-BR-encapsulated CWO NPs (PEG-BR/CWO NPs) were then visualized using TEM with 2% uranyl formate as a negative stain. A representative image of filtered PEG-BR/CWO NPs is shown in FIG. 2.

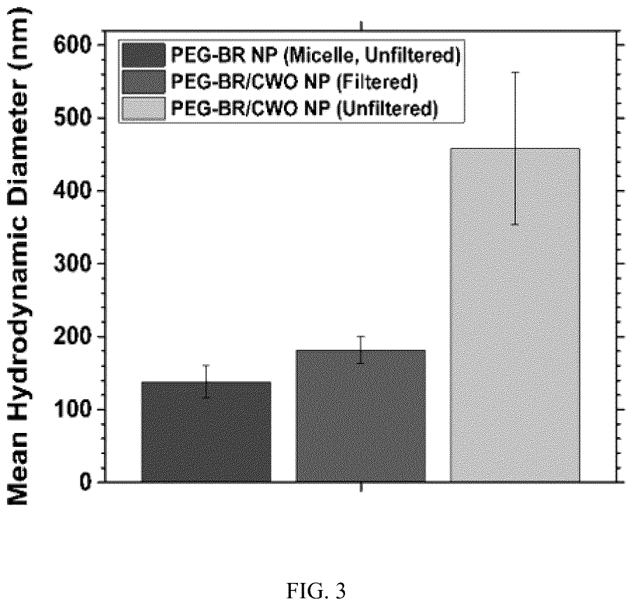

The sizes of PEG-BR micelles and PEG-BR/CWO NPs were characterized via dynamic light scattering (DLS). These results are consistent with the idea that PEG-BR micelles effectively encapsulate CWO nanoparticles. Note that the unfiltered PEG-BR/CWO NPs' effective diameter was larger likely due to large agglomerates of PEG-BR/CWO NPs that may have been present in the sample before filtration. The results of the DLS size measurements are shown in FIG. 3.

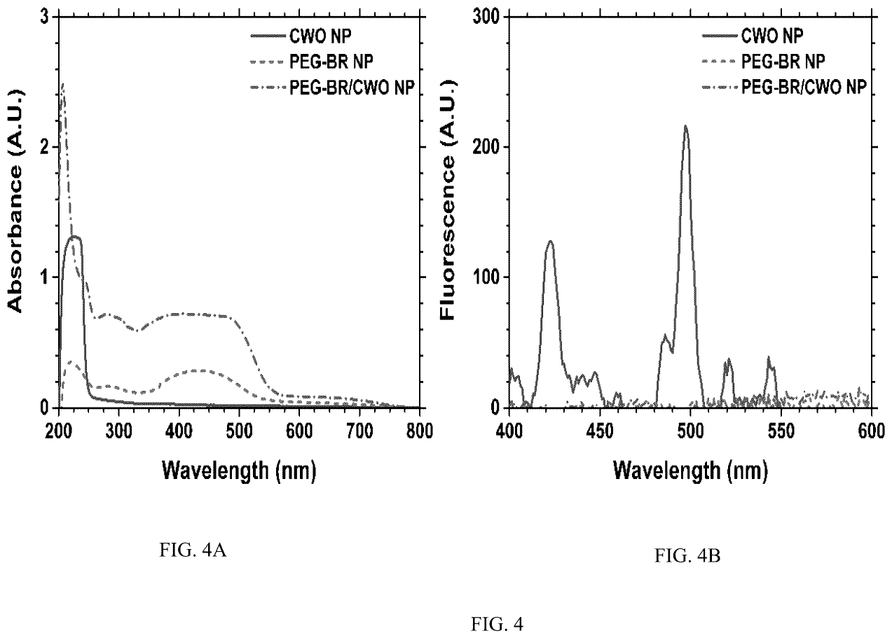

Absorbance and fluorescence measurements were performed on CWO NPs, PEG-BR NPs, and PEG-BR/CWO NPs. A peak absorbance of CWO NPs was observed at around 200 nm; thus this wavelength was used as the excitation wavelength for the fluorescence spectra also shown in FIG. 4. Main fluorescence peaks for CWO NPs were observed at 420 and 495 nm. These wavelengths coincide with the broad absorbance band of PEG-BR NPs. Consequently, no fluorescence was detected from PEG-BR/CWO NPs under 200 nm excitation because the CaWO₄ fluorescence was effectively quenched (absorbed) by nearby bilirubin moieties. This result confirmed that CWO NPs are indeed fully encapsulated by PEG-BR molecules.

### Effects of UV-A/X-Ray Radiation on PEG-BR/CWO NP Morphologies

DLS size measurements of filtered PEG-BR/CWO NPs were conducted before and after exposure to UV-A radiation from a lamp (365 nm peak wavelength) or X-ray irradiation from a stationary anode X-ray generator (320 kV peak energy) to confirm that UV-A/X-ray exposure can cause degradation of the PEG-BR molecules encapsulating the CWO nanoparticles. UV-A exposure (0.56 J/cm²) leads to an increase in effective diameter for the PEG-BR/CWO NP sample, and the size increased again after an additional (subsequent) UV-A dose (0.56 J/cm²). The increase in the effective diameter in this sample is caused by the agglomeration of bare BR-coated CWO nanoparticles that are exposed when the PEG chains dissociate. This data supports the proposed mechanism of action of PEG-BR/CWO NPs (FIG. 1). Exposure of filtered PEG-BR/CWO NPs to 8 Gy X-rays (at a dose rate of 2 Gy/min) was found to cause a greater degree of agglomeration, likely because of a greater degradation of PEG-BR and detachment of PEG under 8 Gy X-ray radiation; the UV-A and X-ray dose values (0.56 J/cm² and 8 Gy, respectively) were chosen because CWO NPs produce about 0.56 J/cm² UV-A light under 8 Gy X-rays.

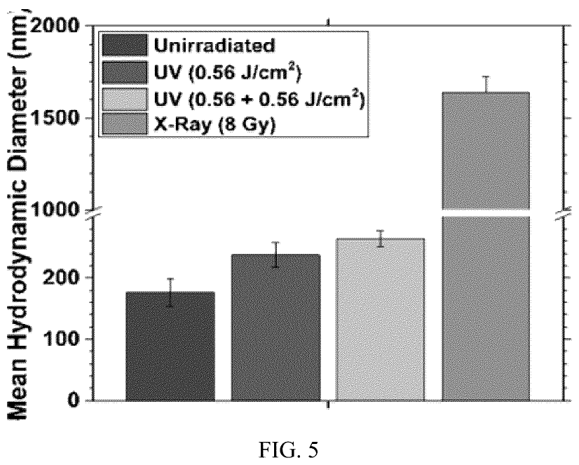

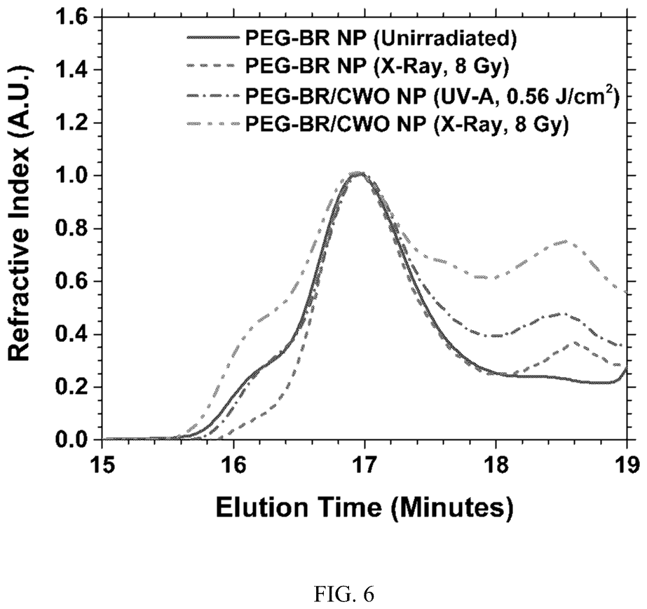

To investigate the cause of the radiation-induced agglomeration of PEG-BR/CWO NPs, irradiated PEG-BR samples were analyzed by GPC. As seen in FIG. 6, both UV-A and X-rays caused the degradation of PEG-BR in PEG-BR/CWO NPs; polymer residues extracted from irradiated PEG-BR/CWO NPs exhibited secondary peaks (shoulders) at about 17.6 and 18.5 minutes of elution time, which suggests that UV-A/X-rays cause the degradation of PEG-BR back to the PEG and BR precursors (GPC traces for the PEG and BR precursors showed peaks at these respective elution times). Notably, the polymer degradation was significantly greater with 8 Gy X-rays than with 0.56 J/cm² UV-A, which is consistent with the greater agglomeration of PEG-BR/CWO NPs observed with 8 Gy X-rays (FIG. 5). The reason for this trend is because, when PEG-BR/CWO NPs are irradiated with X-rays, in addition to UV-A light generated by CWO NPs, X-rays themselves also contribute to the degradation of PEG-BR, as demonstrated in FIG. 6 (i.e., even in the absence of CWO NPs, X-rays cause degradation of PEG-BR). Overall, these data confirm that UV-A/X-ray radiation indeed causes the degradation of PEG-BR in PEG-BR/CWO NPs, resulting in non-PEGylated BR/CWO NPs that agglomerate, as detected by DLS (FIG. 5).

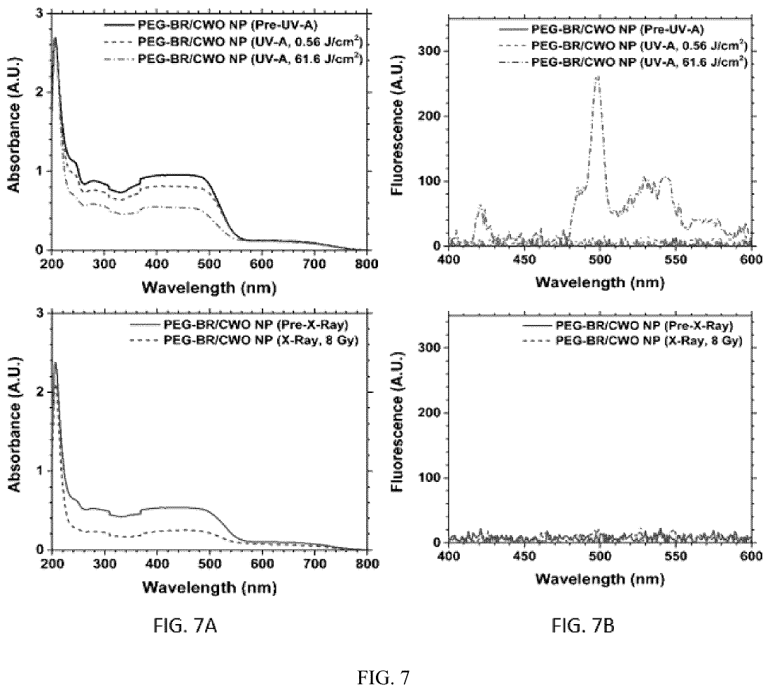

To determine whether the cleaved BR residues remains on the CWO NP surface after the radiation-induced degradation of PEG-BR in PEG-BR/CWO NPs, the absorbance and fluorescence spectra of PEG-BR/CWO NPs were measured before and after exposure to UV-A light (365 nm, 0.56 J/cm²) or X-rays (320 kV, 8 Gy, 2 Gy/min). Neither 0.56 J/cm² UV-A nor 8 Gy X-ray radiation altered the fluorescence-quenched character of the original PEG-BR/CWO NPs, which indicates that the BR monolayer remains adsorbed to the CWO NP surface after the PEG chains are split from the BR moieties (FIG. 6). It has been reported in the literature that the absorption of 450 nm blue light (˜0.6 J/cm²) by PEG-BR disrupts intramolecular hydrogen bonds that cause BR to act as a hydrophobic molecule, and as a result, PEG-BR micelles dissociate into free PEG-BR chains. In our case, neither UV-A nor X-rays rendered BR to become hydrophilic and dissociate from the CWO NP surface. This discrepancy is attributed to the difference in the wavelength of UV-A/blue light used. To validate this explanation, the fluorescence measurements were repeated on PEG-BR/CWO NPs after exposure to a much higher UV-A dose (61.6 J/cm²). As shown in FIG. 7, this excessive UV-A dose caused a recovery of the original fluorescence signals of uncoated CWO NPs (FIG. 4), suggesting that high UV-A doses can indeed eliminate the hydrophobicity of BR. Results from the same sets of experiments with PEG-BR micelles and uncoated CWO NPs confirmed that any of the trends observed in FIG. 7 are not due to any changes in the inherent absorbance/fluorescence characteristics of PEG-BR or CaWO₄ themselves that occur due to exposure to UV-A/X-ray radiation.

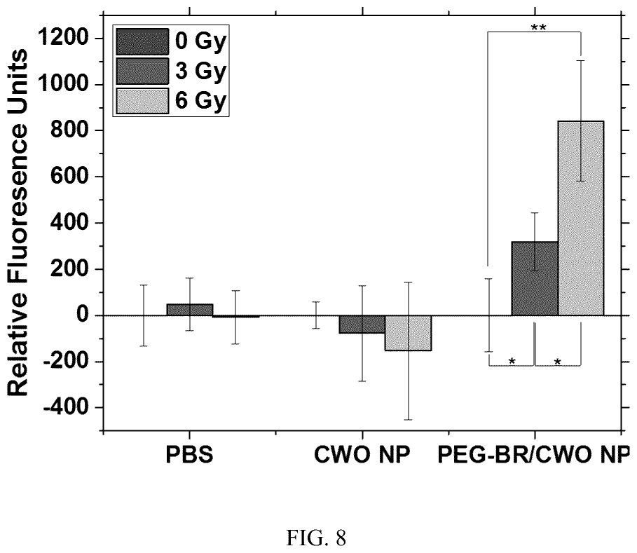

To further explore the mechanism of PEG-BR/CWO NPs, an experiment was conducted to quantify and compare the generation of singlet oxygen (¹O₂), a specific type of reactive oxygen species produced via a type II photosensitizer reaction with molecular oxygen. Relative amounts of Singlet Oxygen Sensor Green (SOSG) fluorescence were compared for PBS, CWO NPs, and PEG-BR/CWO NPs after X-ray radiation at several doses. The results of this experiment are displayed in FIG. 8. The data from the plot suggest that PEG-BR/CWO NPs efficiently generate singlet oxygen in response to X-ray irradiation and do so at an elevated level when compared to PBS or CWO NPs in combination with X-rays. The data also suggest that singlet oxygen production is minimal in the presence of CWO NPs or PBS, indicating that BR-PEG is essential for the photodynamic production of singlet oxygen.

### Biological Evaluation of PEG-BR/CWO NPs

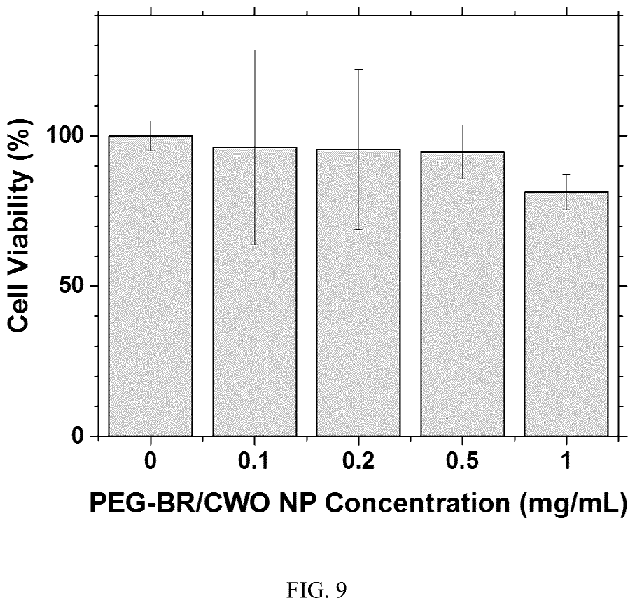

The proposed mechanism for PEG-BR/CWO NPs relies on the idea that the nanoparticles are only activated when illuminated. It then follows that once PEG-BR/CWO NPs are intratumorally injected, only X-ray radiation should be capable of activating the therapeutic effects of the particles. By preventing unwanted activation of NPs, this system is designed to mitigate off-target toxicity. The non-toxic character of uncoated CWO NPs has previously been verified. To examine the extent to which PEG-BR/CWO NPs are cytotoxic in the “dark” (i.e., un-irradiated) state, an MTT cell viability assay was conducted at various concentrations. The results of this experiment are displayed in FIG. 9. As seen in FIG. 9, cell viability remains high until reaching a concentration about an order of magnitude higher than used for therapeutic cell culture treatments (0.1-0.2 mg/mL vs. 1.0 mg/mL). This supports the idea that PEG-BR/CWO NPs are minimally toxic at standard treatment concentrations.

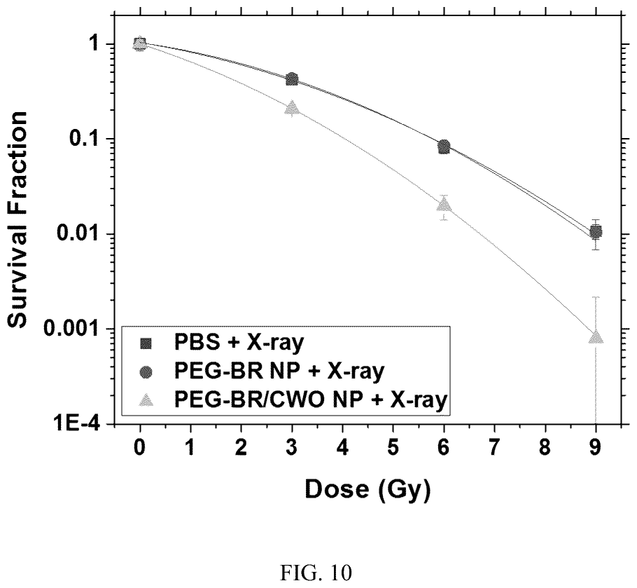

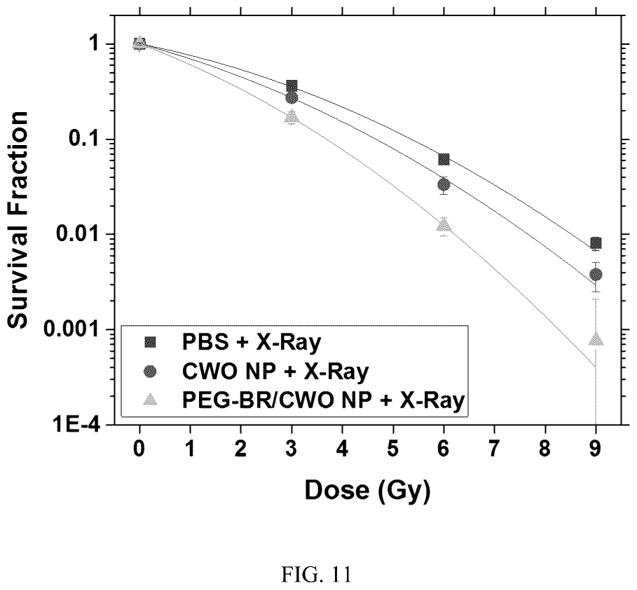

Next, a series of clonogenic cell survival assays were conducted to examine and compare the efficacy of X-ray radiation alone versus X-ray radiation in combination with PEG-BR NPs (micelles), CWO NPs, and PEG-BR/CWO NPs. As shown in FIG. 10 and FIG. 11, a clear increase in cell killing efficacy was observed with PEG-BR/CWO NPs+X-ray relative to all other treatment groups. PEG-BR micelles+X-ray did not produce any increased efficacy compared to X-ray alone, and CWO NPs+X-ray did show enhanced efficacy, as previously observed, but this improvement was not as large as that for PEG-BR/CWO NPs. The sensitizer enhancement ratio (SER) values at 10% cell survival for CWO NPs and PEG-BR/CWO NPs were 1.15 and 1.40, respectively. In addition, the α/β value increased for CWO NPs and PEG-BR/CWO NPs, but this value was also higher for PEG-BR/CWO NPs. These results indicate that CWO NPs alone do not photosensitize cells as significantly as PEG-BR/CWO NPs, and BR-PEG-encapsulation is essential for CWO NPs to mediate photodynamic therapy. These results support the proposed mechanism of action of PEG-BR/CWO NPs and provided motivation for further study in vivo.

According to FIG. 10, Table 1 displays the parameters for the linear-quadratic model fits (SF=exp(αD+βD²), where SF is survival fraction, D is radiation dose, and α and β are fitted parameters) and sensitizer enhancement ratios (SERs) at 10% survival fraction. Note: Experiment conducted using unfiltered particles.

TABLE 1

| | SER | α | β | α/β |
|---|---|---|---|---|
| PBS + X-ray | 1 | −0.200 | −0.035 | 5.7 |
| PEG-BR NP + X-Ray | 1.00 | −0.175 | −0.040 | 4.4 |
| PEG-BR/CWO NP + X-Ray | 1.39 | −0.391 | −0.044 | 8.9 |

According to FIG. 11, Table 2 displays the parameters for the linear-quadratic model fits (SF=exp(αD+βD²), where SF is survival fraction, D is radiation dose, and α and β are fitted parameters) and sensitizer enhancement ratios (SERs) at 10% survival fraction. Note: Experiment conducted using unfiltered particles.

TABLE 2

| | SER | α | β | α/β |
|---|---|---|---|---|
| PBS + X-ray | 1 | −0.243 | −0.035 | 6.9 |
| CWO NP + X-Ray | 1.15 | −0.330 | −0.035 | 9.4 |
| BR-RLNP + X-Ray | 1.40 | −0.455 | −0.046 | 9.9 |

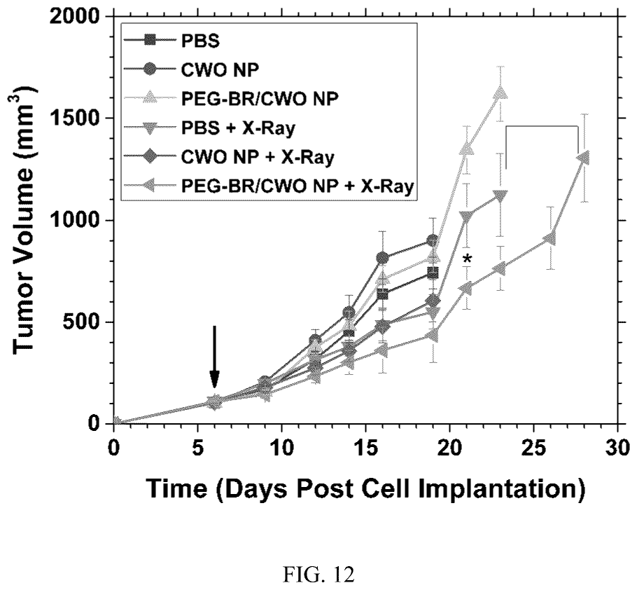

Cell culture experiments screening for safety and efficacy of PEG-BR/CWO NPs provided ample motivation for further study in animal models of head and neck cancer, as mentioned previously. To explore if PEG-BR/CWO NPs exhibited similar efficacy enhancement in vivo, an HN31 xenograft study in Nod rag gamma (NRG) mice was conducted. For this experiment, 8 mice per treatment group had subcutaneous xenografts of HN31 cells, with 6 total treatment groups examined: PBS, CWO NPs, and PEG-BR/CWO NPs±X-ray. Mice received intratumoral injections of 10 mg/mL (based on CWO NP concentration) or PBS split into two equal doses on days 6 and 7 of the study post HN31 inoculation (day 0). Total X-ray dose used was 4 Gy split over two consecutive fractions (2+2 Gy on days 7 and 8). Mouse tumor volumes for each treatment group over time are displayed in FIG. 12, plotted up to the first euthanasia event for each treatment group. On day 21, the PBS+X-ray and PEG-BR/CWO NP+X-ray groups were sufficiently separated to reach statistical significance (p<0.1).

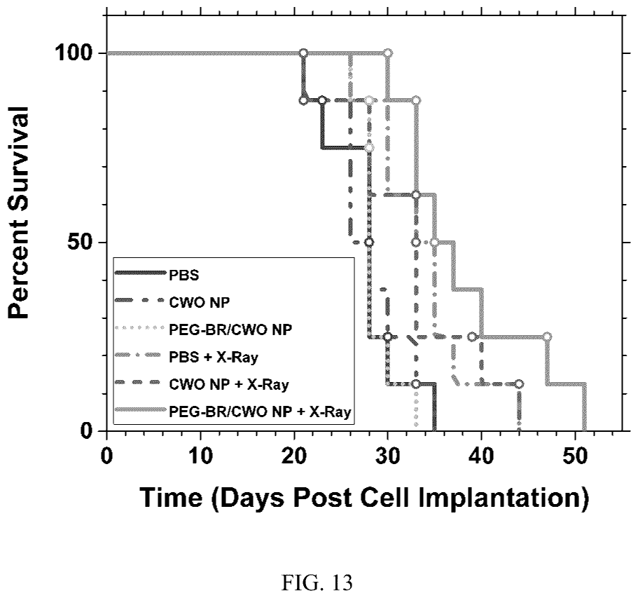

FIG. 13 displays the mouse survival over time for each treatment group. As seen in FIG. 13, the median survival times for the PEG-BR/CWO NP+X-Ray, CWO NP+X-Ray, and PBS+X-Ray groups were 35, 33, and 33 days post-cell implantation, respectively. One-way ANOVA testing was conducted to determine if a significant difference in group survival existed. Each irradiated treatment group (the “+X-Ray” groups) was independently tested against its respective un-irradiated controls, and each was found to be significantly different within their pair except for CWO NP±X-ray. However, when the irradiated groups were compared with each other, none of the groups were significantly different from each other, though PEG-BR/CWO NP+X-Ray was somewhat close to reaching a p-value of less than 0.1 (p=0.139). The results of ANOVA testing are displayed in Table 3.

TABLE 3

| | | | PEG-BR/ | | | PEG-BR/ |
|---|---|---|---|---|---|---|
| | PBS | CWO NP | CWO NP | PBS + X-Ray | CWO NP + X-Ray | CWO NP + X-Ray |
| PBS | — | 0.82145 | 0.55834 | 0.02496 | 0.12085 | 0.00324 |
| CWO NP | — | — | 0.77754 | 0.04015 | 0.16559 | 0.00492 |
| PEG-BR/CWO NP | — | — | — | 0.02600 | 0.16506 | 0.00309 |
| PBS + X-Ray | — | — | — | — | 0.70098 | 0.18556 |
| CWO NP + X-Ray | — | — | — | — | — | 0.13590 |

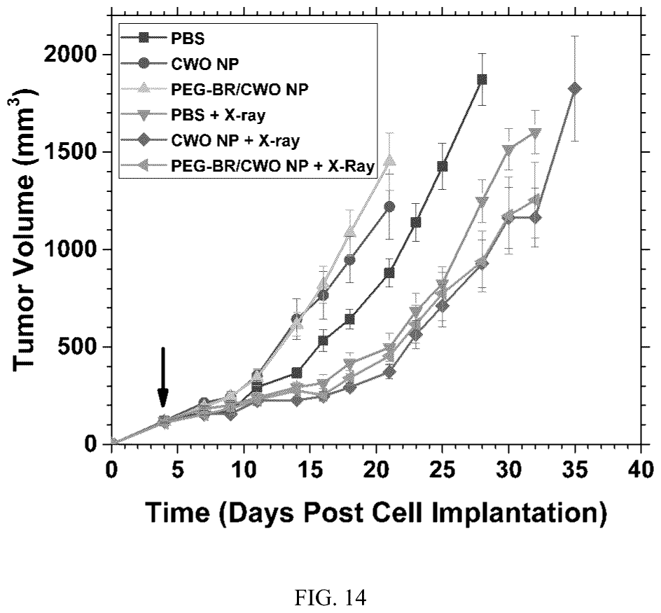

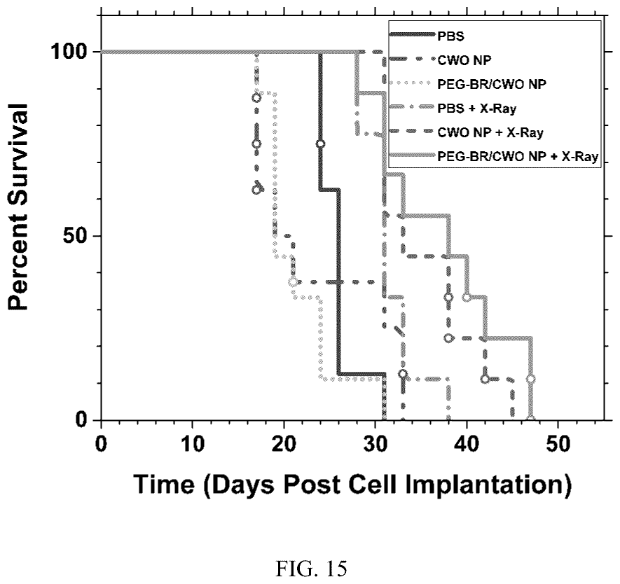

The in vivo tumor growth and mouse survival tests were repeated with an increased total X-ray dose of 8 Gy (split over 4 consecutive fractions given in 2 Gy per fraction per day). The results are presented in FIG. 14 and FIG. 15. Interestingly, both PEG-BR/CWO NPs and uncoated CWO NPs produced comparable levels of radiotherapy enhancement in tumor growth suppression (FIG. 14). However, similarly to the 4 Gy situation, the therapeutic benefit of PEG-BR/CWO NPs (relative to CWO NPs) was better manifested in terms of mouse survival time (FIG. 15); the median survival times for PEG-BR/CWO NP+X-ray, CWO NP+X-ray, and PBS+X-ray groups were 38, 33, and 31 days post cell implantation, respectively. The mouse survival data were analyzed by one-way ANOVA. As shown in the Table 4, all irradiated groups exhibited significant increases in survival time relative to their respective unirradiated control groups. Notably, PEG-BR/CWO NP+X-ray was significantly different from PBS+X-Ray (p=0.036), whereas CWO NP+X-Ray was not (p=0.069). Also, the difference between PEG-BR/CWO NP+X-Ray versus CWO NP+X-Ray was again not statistically significant (p=0.53), although the order of therapeutic effectiveness among the X-ray-treated groups was reproducible between the 4 Gy and 8 Gy studies: PEG-BR/CWO NP+X-Ray>CWO NP+X-Ray>PBS+X-Ray. Taken together, these conclusions indicate that doubling the total radiation dose did in fact result in a greater degree of sensitizer-enhancement in vivo.

TABLE 4

| | | | PEG-BR/ | | | PEG-BR/ |
|---|---|---|---|---|---|---|
| | PBS | CWO NP | CWO NP | PBS + X-Ray | CWO NP + X-Ray | CWO NP + X-Ray |
| PBS | — | 0.404161 | 0.020274 | 0.000589 | 0.000283 | 0.000516 |
| CWO NP | — | — | 0.49088 | 0.009223 | 0.001567 | 0.001287 |
| PEG-BR/CWO NP | — | — | — | 2.82E−05 | 1.43E−05 | 2.78E−05 |
| PBS + X-Ray | — | — | — | — | 0.069536 | 0.035659 |
| CWO NP + X-Ray | — | — | — | — | — | 0.533566 |

The lack of significant difference between irradiated group survival times (and of most of the irradiated tumor volumes, for that matter) is believed to be due to the limited number of radiation fractions administered to treat the mice (2 or 4 fractions of 2 Gy=4 or 8 Gy total). At low doses of radiation (i.e., 2 Gy), the difference in survival fraction between PBS+X-ray, CWO NP+X-ray, and PEG-BR/CWO NP+X-ray-treated cells is small (FIG. 11). Thus, over the course of a typical radiotherapy prescription of 25-30 fractions of 2 Gy, a clear difference in tumor cell death will emerge. In this mouse study, however, with only 2 or 4 fractions of radiation, the difference in cell death in vivo is not large enough to manifest in significant survival benefits.

This point can further be explained by using a simple theoretical argument as follows. Using the linear-quadratic model parameters (α and β) obtained from in vitro clonogenic assays (FIG. 5), the values of survival fraction (SF) of HN31 cells after irradiation with 8 Gy X-rays are estimated to be: SF (D=2 Gy)=0.335, 0.449 and 0.535 for the PEG-BR/CWO NP+X-ray, CWO NP+X-ray and PBS+X-ray groups, respectively. If n fractions of 2 Gy per fraction are applied, the survival fractions of HN31 cells can be predicted by SF (D=n×2 Gy)≈[SF(D=2 Gy)]ⁿ, assuming that the time interval between radiation fractions (1 day) was sufficient for cell's recovery from sub-lethal radiation damage. In in vivo studies presented in FIG. 13 and FIG. 15, HN31 xenografts were treated with X-rays when individual tumors reached about 0.10 cc in volume (≈10⁸ cells assuming a cell density of ρₒ≈10⁹ cells per cc of tumor). Therefore, in our in vivo studies, the number of clonogenically active cells within the tumor immediately following 4 fractions (n=4) of 2 Gy radiation (at Day 8 in FIG. 14) is estimated to be: Nₒ≈1.26×10⁶, 4.06×10⁶ and 8.19×10⁶ cells for PEG-BR/CWO NP+X-Ray, CWO NP+X-Ray and PBS+X-Ray, respectively. From FIG. 14 (i.e., from the slopes of the tumor growth curves between Days 0 and 17), the in vivo doubling times of HN31 cells are estimated to be: t₂≈4.61, 4.89 and 5.87 days for the PEG-BR/CWO NP-, CWO NP- and PBS-treated xenografts, respectively. Therefore, the mouse survival time (tₛ defined as the time it takes for the irradiated tumor to reach the euthanasia threshold in volume (V_{f}≈2.0 cc)) post 4×2 Gy radiation can be estimated by:

$${t}_{s} = {t}_{2} \frac{ln ( {V}_{f} {ρ}_{0} / {N}_{o} )}{ln ( 2 )} . \qquad ( 1 )$$

The predicted values of the mouse survival times in the 8 Gy study are: tₛ≈47.9, 42.6 and 45.2 days post radiation for PEG-BR/CWO NP+X-Ray, CWO NP+X-Ray and PBS+X-Ray, respectively. Similar calculations have also been performed for the 4 Gy study. As shown in Table 5, the predictions are in reasonable agreement with experimental results despite the simplistic, deterministic nature of the theoretical model. Using the above SF model, it is possible to predict mouse survival times under dose conditions close to clinical practice. The numbers of clonogenically active cells within the tumor immediately following, for instance, 30 fractions (n=30) of 2 Gy radiation are estimated to be: Nₒ≈5.64×10⁻⁷, 8.77×10⁻² and 7.09×10⁻¹ cells for PEG-BR/CWO NP+X-Ray, CWO NP+X-Ray and PBS+X-Ray, respectively. Using the same procedure as above, the survival times are estimated to be: tₛ≈238, 168 and 184 days post radiation for PEG-BR/CWO NP+X-Ray, CWO NP+X-Ray and PBS+X-Ray, respectively. Concurrent PEG-BR/CWO NP+X-Ray is, therefore, predicted to produce a significant survival benefit of about 2 months relative to both X-rays only (i.e., PBS+X-Ray) and CWO NP+X-Ray.

TABLE 5
Predicted versus Measured Median Mouse Survival Times. Survival time predictions
were made based on in vitro cell survival fractions (from FIG. 11). Details
on calculations and assumptions can be found in the main discussion.

| | | | | Predicted Mouse | Measured Median |
|---|---|---|---|---|---|
| | | Cell | Tumor | Survival Time | Mouse Survival |
| | X-Ray Dose | Survival | Doubling Time | (Days Post | Time (Days Post |
| Treatment Group | (Gy) | Fraction | (Days) | Radiation) | Radiation) |
| PBS | 4 | 0.2859 | 4.25 | 26.0 | 26 |
| | 8 | 0.0818 | 5.87 | 45.2 | 23 |
| CWO NP | 4 | 0.2019 | 3.85 | 25.5 | 26 |
| | 8 | 0.0408 | 4.89 | 42.6 | 25 |
| PEG-BR/CWO NP | 4 | 0.1121 | 4.05 | 30.3 | 28 |
| | 8 | 0.0126 | 4.61 | 47.9 | 30 |

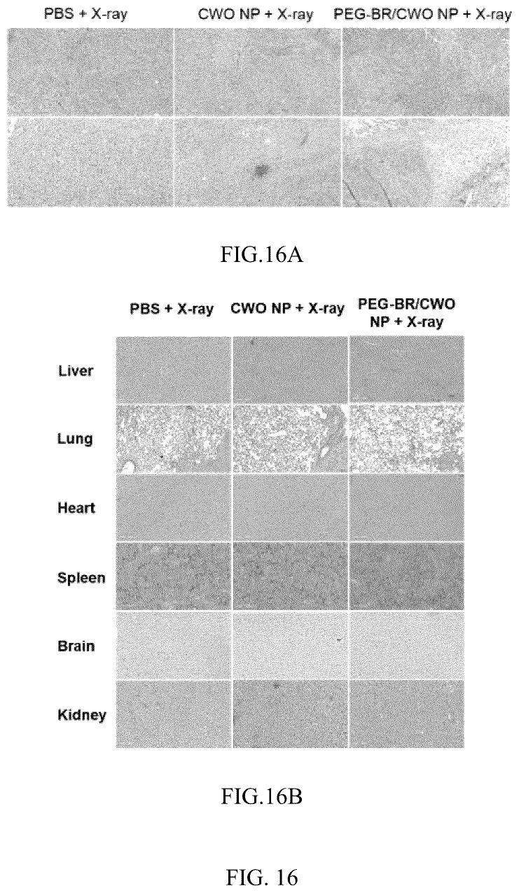

Finally, histology slides of major organ and tumor tissues were prepared for the PBS+X-Ray, CWO NP+X-Ray and PEG-BR/CWO NP+X-ray groups, and stained using hematoxylin and eosin (H&E). This was conducted to compare major organ tissues between treatment groups and to examine the condition of the tumor after exposure to each treatment. Representative images of the major organs and tumors from each group are displayed in FIG. 16 (total 4 Gy-treated groups). As seen in the figure, major organ tissue sections appear nearly identical between treatment groups. Lung metastases and enlarged spleens were observed for each of these treatment groups, and histopathological evidence of metastases are displayed in each of the lung images presented (FIG. 16B, deep purple nodules in the corner of each image). Tumor section comparisons display two images taken from different regions of the tumor. The top images were taken from areas of relatively high tumor cell viability (evidenced by consistent purple staining and morphology) with interspersed necrotic regions. The bottom images were taken from areas of high damage within the tumor sections, evidenced by widespread necrotic regions, interspersed gaps in tissue, and lack of nuclei or dense tissue altogether. PEG-BR/CWO NP+X-ray-treated tumor showed a slightly larger region of low numbers of cell nuclei and lack of dense tissue when compared to the other treatment groups. These data suggest that PEG-BR/CWO NPs do not disperse or damage major organs following intratumoral administration and lead to enhanced necrosis/mitotic arrest within treated tumors.

We have demonstrated that PEG-BR/CWO NPs can be used to potentiate photodynamic therapy under X-ray irradiation. In PEG-BR/CWO NPs, CWO NPs are encapsulated within a capsule formed by PEG-BR molecules. We further explored whether it is possible to further load chemo drugs (such as paclitaxel (PTX)) within PEG-BR/CWO NPs. Such formulation (which we will name as “PEG-BR/CWO/PTX NPs”) will enable us to combine three therapeutic modalities in one regimen: radiotherapy, photodynamic therapy, and chemotherapy.

PEG-BR/CWO/PTX NPs could be produced by co-encapsulating CWO NPs and PTX within a PEG-BR micelle via solvent exchange (as described in Materials and Methods). As summarized in Table 6, the PTX loading efficiency (defined as the mass of PTX encapsulated divided by the mass of PTX initially added) was found to be about 2%. This number means that each primary CWO NP (of about 45 nm diameter) is surrounded by a coating layer consisting approximately of 2.8×10⁻¹⁷ g of PEG-BR and 1.4×10⁻¹⁶ g of PTX.

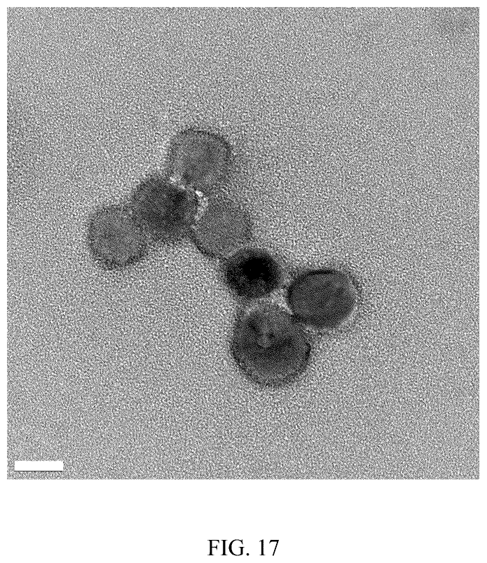

PEG-BR/CWO/PTX NPs were visualized by TEM (FIG. 17). As shown in the figure, the encapsulated PTX appears to produce a dark ring around each primary CWO NP. The size characteristics of PEG-BR/CWO/PTX NPs were characterized by DLS. The mean hydrodynamic diameters were measured to be about 487 and 158 nm before and after filtration with a 450 nm PTFE syringe filter, respectively.

Table 6. Loading of PTX within PEG-BR/CWO NPs. PTX-co-loaded PEG-BR-encapsulated CWO NPs (“PEG-BR/CWO/PTX NPs”) were prepared using the same procedure as for PEG-BR/CWO NPs, except that PTX was initially co-dissolved with PEG-BR in DMF prior to solvent exchange with PBS. The amounts of PTX and PEG-BR dissolved in 3.9 mL of DMF were varied as shown in the table. After solvent exchange, PEG-BR/CWO/PTX NPs were diluted with PBS to 0.25 mg/mL (based on mass of CWO), centrifuged, and re-suspended in PBS to a CWO concentration of 0.25 mg/mL. In order to determine the encapsulated amount of PTX by HPLC, 2.0 mL of dichloromethane (DCM) was added to 2.0 mL of the PEG-BR/CWO/PTX NP solution in PBS. The mixture was vortexed to extract PTX into the DCM phase. The emulsion was then centrifuged, and the bottom DCM layer was collected and dried overnight. The dried residue was re-dissolved in 200 μL of HPLC-grade acetonitrile (ACN) for HPLC analysis. Reversed phase HPLC was carried out using a Zorbax C-18 5-μm column with a water/ACN (45:55 by volume) mixture as the mobile phase at a flowrate of 1 mL/min. Each sample was run for 7 minutes with PTX eluting at ˜5 minutes. The concentration of PTX was estimated from the area of the peak in comparison with a predetermined calibration curve of peak area vs. concentration. The loading efficiency was calculated as the mass of PTX encapsulated divided by the mass of PTX initially added.

TABLE 6

| Mass of | Mass of | Mass of PTX | PTX Loading |
|---|---|---|---|
| PEG-BR Added | PTX Added | Encapsulated | Efficiency |
| (mg) | (mg) | (mg) | (%) |
| 20 | 2 | 0.0425 | 2.12 |
| 20 | 4 | 0.0749 | 1.87 |
| 20 | 6 | 0.0953 | 1.59 |

Taken together, this study provides ample data that suggest PEG-BR/CWO NPs are a novel formulation that can mediate combined radio/photodynamic therapy in solid tumors. The results demonstrate the new use of PEG-BR micelles as an encapsulant for CaWO₄ nanoparticles. PEG-BR/CWO NPs emit UV-A and visible light under X-ray that causes degradation of their PEG-BR encapsulant and subsequent dissociation of the free PEG chains, allowing for the continued excitation of the now-water-exposed bilirubin by the UV-A/visible light. This key step initiates the photodynamic therapy response by producing reactive oxygen species like singlet oxygen which complement the lethal effects of X-rays to enhance cancer cell death. In vitro efficacy testing demonstrated clear therapeutic enhancements in combining PEG-BR/CWO NPs with X-ray radiotherapy. Furthermore, a head and neck cancer xenograft experiment in mice suggested that these combined radio/photodynamic therapy enhancements are present in vivo. PEG-BR/CWO NPs represent a novel platform for combining radiation and photodynamic (and even chemo) therapies for solid tumors, and further optimization and efficacy validation are warranted to examine their ultimate translational viability.

Those skilled in the art will recognize that numerous modifications can be made to the specific implementations described above. The implementations should not be limited to the particular limitations described. Other implementations may be possible.

We claim:

1. A method of treating a disease responsive to a composition comprising:
a radio-luminescent particle or particle aggregate; and
a hydrophilic polymer-conjugated bilirubin;
wherein the radio-luminescent particle or particle aggregate is encapsulated in the hydrophilic polymer-conjugated bilirubin
wherein the method comprises administering the composition directly into the diseased site and exposing the diseased site to an ionizing radiation, wherein the ionizing radiation comprises UV light, X-rays, γ rays, electrons, protons, neutrons, ions, or any combination thereof.

2. The method of claim 1, wherein the disease is a cancer.

3. The method of claim 1, wherein the radio-luminescent particle or particle aggregate is a metal tungstate material (Mₓ(WO₄)_{y}) or a metal molybdate material (Mₓ(MoO₄)_{y}), wherein a metal compound (M) is selected from the “Alkaline Earth Metal”, “Transition Metal” or any combination thereof.

4. The method of claim 3, wherein the radio-luminescent particle or particle aggregate is selected from calcium tungstate (CaWO₄), iron tungstate (FeWO₄), manganese tungstate (MnWO₄), calcium molybdate (CaMoO₄), iron molybdate (FeMoO₄), manganese molybdate (MnMoO₄), and a combination of two or more thereof.

5. The method of claim 1, wherein the radio-luminescent particle or particle aggregate is zinc oxide (ZnO), zinc sulfide (ZnS), or a combination thereof.

6. The method claims 1, wherein the hydrophilic polymer-conjugated bilirubin forms a self-assembled structure in water, wherein the radio-luminescent particle or particle aggregate is encapsulated within the hydrophobic subdomain of the self-assembled structure formed by the bilirubin component.

7. The method of any claim 1, wherein the hydrophilic polymer comprises a monomer selected from the group consisting of ethylene glycol, ethylene oxide, vinyl alcohol, oxazoline, acrylic acid, methacrylic acid, acrylamide, styrene sulfonate, saccharide, imine, vinyl pyrrolidone, vinyl pyridine, and lysine.

8. The method of claim 1, wherein the hydrophilic polymer-conjugated bilirubin is poly (ethylene glycol) (PEG)-conjugated bilirubin.

9. The method of claim 1, wherein the composition further comprises a hydrophobic chemotherapeutic drug, wherein the radio-luminescent particle or particle aggregate and the hydrophobic chemotherapeutic drug are co-encapsulated within a capsule formed by the hydrophilic polymer-conjugated bilirubin, wherein the hydrophobic chemotherapeutic drug comprises paclitaxel, docetaxel, cabazitaxel, cisplatin, carboplatin, oxaliplatin, nedaplatin, doxorubicin, daunorubicin, epirubicin, idarubicin, gemcitabine, etanidazole, 5-fluorouracil, methotrexate, any salt or derivative thereof, or any combination thereof.

10. A method of treating a patient with locally advanced primary or metastatic tumors, wherein the method comprises administering to the pateint a therapeutically effective amount of composition into the primary or metastatic tumors and exposing the tumors to an ionizing radiation, wherein the composition comprises:
a radio-luminescent particle or particle aggregate; and
a hydrophilic polymer-conjugated bilirubin;
wherein the radio-luminescent particle or particle aggregate is encapsulated with the hydrophilic polymer-conjugated bilirubin.

11. The method of claim 10, wherein the ionizing radiation comprises UV light, X-rays, γ rays, electrons, protons, neutrons, ions, or any combination thereof.

12. The method of claim 10, wherein said tumors are solid tumors.

13. The method of claim 12, wherein said tumors are related to head and neck, lung, brain, muscle, bone, stomach, liver, pancreatic, renal, colon, rectal, prostate, breast, gynecological, or cervical tissues.

14. The method of claim 10, wherein the composition further comprises a hydrophobic chemotherapeutic drug, wherein the radio-luminescent particle or particle aggregate and the hydrophobic chemotherapeutic drug are co-encapsulated within a capsule formed by the hydrophilic polymer-conjugated bilirubin, wherein the hydrophobic chemotherapeutic drug comprises paclitaxel, docetaxel, cabazitaxel, cisplatin, carboplatin, oxaliplatin, nedaplatin, doxorubicin, daunorubicin, epirubicin, idarubicin, gemcitabine, etanidazole, 5-fluorouracil, methotrexate, any salt or derivative thereof, or any combination thereof.

15. The method of claim 10, wherein the radio-luminescent particle or particle aggregate is selected from calcium tungstate (CaWO₄), iron tungstate (FeWO₄), manganese tungstate (MnWO₄), calcium molybdate (CaMoO₄), iron molybdate (FeMoO₄), manganese molybdate (MnMoO₄), and a combination of two or more thereof.

16. The method of any claim 10, wherein the hydrophilic polymer comprises a monomer selected from the group consisting of ethylene glycol, ethylene oxide, vinyl alcohol, oxazoline, acrylic acid, methacrylic acid, acrylamide, styrene sulfonate, saccharide, imine, vinyl pyrrolidone, vinyl pyridine, and lysine.

17. The method of claim 1, wherein the hydrophilic polymer-conjugated bilirubin is poly (ethylene glycol)(PEG)-conjugated bilirubin.
